(19) United States
(12) Patent Application Publication
(10) Pub. No.: US 2019/0021601 A1
(43) Pub. Date: Jan. 24, 2019

(54) Compact Imaging System and Method Therefor

(71) Applicant: Colgate-Palmolive Company, New York, NY (US)

(72) Inventor: Hrebesh Molly Subhash, Highland Park, NJ (US)

(73) Assignee: Colgate-Palmolive Company, New York, NY (US)

(21) Appl. No.: 15/654,104

(22) Filed: Jul. 19, 2017

(51) Int. Cl. A61B 5/00; G01B 9/02; G01N 21/47; G01N 21/85; G02B 6/26

(52) U.S. Cl. CPC A61B 5/0066; A61B 5/6847; G01B 9/02091; G01N 21/474; G01N 2021/1787; G01N 21/8507; G02B 6/262; G01B 2290/65; G01B 2290/70; G01N 21/4795

(57) ABSTRACT

An imaging system may include: a scan head optically coupled to the light source and configured to scan light from a light source onto a portion of a specimen and to receive light reflected from the portion of the specimen; an interferometer optically coupled to the light source and to the scan head to receive and generating an interference output using light from the light source and light reflected from the portion of the specimen; a scanning spectrometer optically coupled to the interferometer to receive the interference output and generating a scanned output, the scanned output having a sub-spectrum of light which is narrower than a spectrum of light generated by the light source; and a detector optically coupled to the scanning spectrometer to detect the scanned output and generating a detector signal from the detected scanned output.

### BACKGROUND

As a multifunctional clinical diagnostic and monitoring technique, optical coherence tomography (OCT) has become a well-established tool in many areas of medicine including ophthalmology, dermatology, dentistry, gastrointestinal endoscopy, intravascular imaging, and oncology, among others. OCT is a powerful tool for providing image-based data in real-time in high-resource settings to aid diagnosis, screening, and treatment monitoring. However, there exist several clinical and non-clinical settings, including personal care applications, in which OCT, and the imaging capabilities it provides, would be beneficial. Presently, OCT is implemented using frequency-domain (FD) (such as spectral-domain OCT and swept-source OCT) or time-domain (TD), methods to obtain and interpret the interference signal from biological and non-biological specimens. While each method presents different advantages, existing OCT systems, regardless of the method, are generally bulky and require the addition of expensive optical and electronic peripherals in order to meet the needs of the intended purpose. Furthermore, existing systems tend to require accurate alignment of complex optics, and this requirement can make them highly impractical for point-of-care and personal care environments.

OCT systems which employ FD methods and operate at short wave infrared (SWIR) wavelengths, ranging from about 900 nm to 1800 nm, are superior for subsurface imaging of tissues which cause a high degree of scattering. Such systems are generally less impacted by scattering due to the increased penetration depth of the scanned light. Some OCT systems which employ FD methods use swept-source technology, and such technology requires a highly sophisticated and expensive SWIR laser and a high speed digitizer for implementation.

OCT systems which employ TD methods require a complex interferometer with scanning reference arm to generate the interference signals. Such interferometers can add significantly to the overall expense and complexity of the OCT system.

OCT systems which employ spectral-domain (SD) methods have their own shortcomings. Such systems generally require a bulky and expensive spectrometer combined with a complex InGaAs linear array detector. In addition, an InGaAs linear array detector not only requires a sophisticated cooling system, but it also requires complex preprocessing algorithms to correct for sensor-inherent anomalies that would otherwise be found in the images. Thus, not only are these types of OCT systems bulky, they are also expensive to manufacture and expensive to implement and use.

In view of the impracticality of existing OCT systems for certain environments, there is a need for a cost-effective, compact, and easy to use OCT platform that can readily be used for personal and point-of-care diagnostic applications. Such an OCT platform can enable the rapid and accurate diagnosis and monitoring of patients, while also reducing the cost and time associated with healthcare services.

### BRIEF SUMMARY

Exemplary embodiments according to the present disclosure are directed to imaging systems and methods which employ optical coherence tomography (OCT) to image specimens. The imaging system employs a scan head, an interferometer, and a scanning spectrometer. The scanning spectrometer receives input from the scan head and the interferometer, and signals output from the scanning spectrometer are detected and analyzed to produce an image of the specimen. The system may be used to image any type of specimen, including hard or soft tissues of the human body, and it has further applications for imaging any other type of specimen (e.g., any organic or inorganic solid or semisolid specimen) in a cost-effective manner. The imaging method includes steps of scanning a specimen using a scan head and light from a light source, processing light reflected from the specimen through an interferometer, through a scanning spectrometer, and then detecting light emerging from the scanning spectrometer at a detector to generate a detector signal. The detector signal may then be processed to generate the image of the specimen. The method may also include communicating the generated image to the user in real time.

In one aspect, the invention can be an imaging system including: a scan head optically coupled to the light source and configured to scan light from a light source onto a portion of a specimen and to receive light reflected from the portion of the specimen; an interferometer optically coupled to the light source and to the scan head to receive and generating an interference output using light from the light source and light reflected from the portion of the specimen; a scanning spectrometer optically coupled to the interferometer to receive the interference output and generating a scanned output, the scanned output having a sub-spectrum of light which is narrower than a spectrum of light generated by the light source; and a detector optically coupled to the scanning spectrometer to detect the scanned output and generating a detector signal from the detected scanned output.

In another aspect, the invention can be an imaging method including: scanning light from a light source onto a portion of a specimen; generating an interference output by collinearly coupling light from the light source and reflected light from the portion of the specimen; generating a scanned output using a scanning spectrometer by dispersing the interference output into a plurality of sub-spectrum within the predetermined spectrum, and by cycling through each of the sub-spectrum to form the scanned output; generating a detector signal from the scanned output; process the detector signal to generate an image of the portion of the specimen.

In still another aspect, the invention can be an imaging system including: a data acquisition subsystem including: a light source producing light in a predetermined spectrum; a scan head optically coupled to the light source and configured to scan light from the a light source onto a portion of a specimen and to receive light reflected from the portion of the specimen; an interferometer optically coupled to the light source and to the scan head to receive, respectively, light from the light source and light reflected from the portion of the specimen, the interferometer configured to generate an interference output by collinearly coupling light from the light source and light reflected from the portion of the specimen; a scanning spectrometer optically coupled to the interferometer to receive the interference output and configured to disperse the interference output into a plurality of sub-spectrum within the predetermined spectrum, wherein the scanning spectrometer generates a scanned output by cycling through each of the sub-spectrum; and a detector optically coupled to the scanning spectrometer to detect the scanned output, the detector configured to generate a detector signal from the detected scanned output; and a data processing subsystem including: a processor operatively coupled to the detector to receive the detector signal, the processor programmed to generate an image of the portion of the specimen from the detector signal; and a wireless transceiver operatively coupled to the processor, wherein the processor is programmed to transmit the image using the wireless transceiver.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The foregoing summary, as well as the following detailed description of the exemplary embodiments, will be better understood when read in conjunction with the appended drawings. It should be understood, however, that the invention is not limited to the precise arrangements and instrumentalities shown in the following figures:

FIG. 1 schematically illustrates an imaging system in accordance with a first embodiment of the present invention;

FIG. 2 schematically illustrates an imaging system in accordance with a second embodiment of the present invention;

FIG. 3 schematically illustrates an imaging system in accordance with a third embodiment of the present invention;

FIG. 4 is a flowchart showing a process for imaging a specimen; and

FIGS. 5A-D graphically illustrate an image reconstruction process for imaging an eye according to another embodiment of the invention.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “left,” “right,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the preferred embodiments. Accordingly, the invention expressly should not be limited to such preferred embodiments illustrating some possible non-limiting combinations of features that may exist alone or in other combinations of features; the scope of the invention being defined by the claims appended hereto.

Features of the present invention may be implemented in software, hardware, firmware, or combinations thereof. The programmable processes described herein are not limited to any particular embodiment, and may be implemented in an operating system, application program, foreground or background processes, driver, or any combination thereof. The computer programmable processes may be executed on a single processor or on or across multiple processors.

Processors described herein may be any central processing unit (CPU), microprocessor, micro-controller, computational, or programmable device or circuit configured for executing computer program instructions (e.g. code). Various processors may be embodied in computer and/or server hardware and/or computing device of any suitable type (e.g. desktop, laptop, notebook, tablet, cellular phone, smart phone, PDA, etc.) and may include all the usual ancillary components necessary to form a functional data processing device including without limitation a bus, software and data storage such as volatile and non-volatile memory, input/output devices, a display screen, graphical user interfaces (GUIs), removable data storage, and wired and/or wireless communication interface devices including Wi-Fi, Bluetooth, LAN, etc.

Computer-executable instructions or programs (e.g. software or code) and data described herein may be programmed into and tangibly embodied in a non-transitory computer-readable medium that is accessible to and retrievable by a respective processor as described herein which configures and directs the processor to perform the desired functions and processes by executing the instructions encoded in the medium. A device embodying a programmable processor configured to such non-transitory computer-executable instructions or programs is referred to hereinafter as a “programmable device”, or just a “device” for short, and multiple programmable devices in mutual communication is referred to as a “programmable system”. It should be noted that non-transitory “computer-readable medium” as described herein may include, without limitation, any suitable volatile or non-volatile memory including random access memory (RAM) and various types thereof, read-only memory (ROM) and various types thereof, USB flash memory, and magnetic or optical data storage devices (e.g. internal/external hard disks, floppy discs, magnetic tape CD-ROM, DVD-ROM, optical disk, ZIP™ drive, Blu-ray disk, and others), which may be written to and/or read by a processor operably connected to the medium.

In certain embodiments, the present invention may be embodied in the form of computer-implemented processes and apparatuses such as processor-based data processing and communication systems or computer systems for practicing those processes. The present invention may also be embodied in the form of software or computer program code embodied in a non-transitory computer-readable storage medium, which when loaded into and executed by the data processing and communications systems or computer systems, the computer program code segments configure the processor to create specific logic circuits configured for implementing the processes.

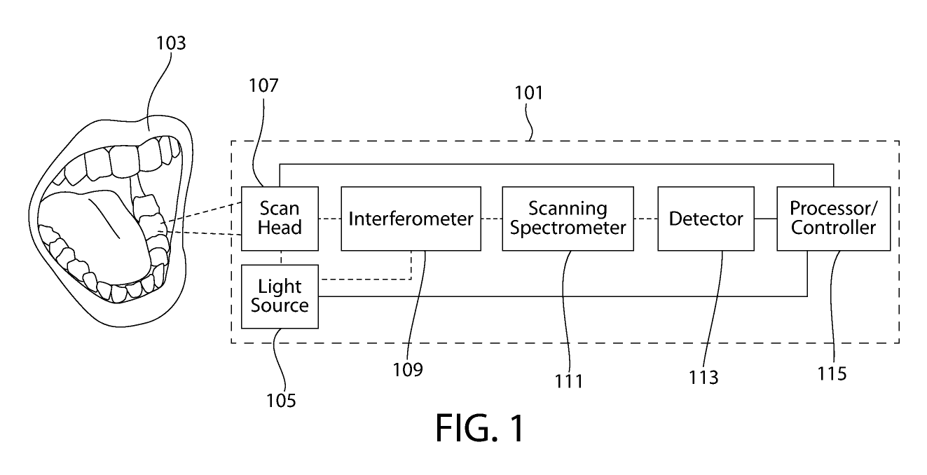

Turning in detail to the drawings, FIG. 1 illustrates an imaging system 101 in accordance with an embodiment of the present invention. The imaging system 101 is able to produce an image of a specimen 103 using an SD technique for optical coherence tomography (OCT). The imaging system 101 is positioned to image the specimen 103, which is shown in FIG. 1 as oral tissue. The imaging system 101 can image both hard and soft oral tissues, and as discussed below, can be used to image other types of tissue, other than oral, and other types of solid or semi-solid materials, including organic and inorganic specimens. The imaging system 101 includes a light source 105 and a scan head 107 optically coupled to each other. In certain embodiments, the light source 105 may be a broadband light source, such as a superluminescent light emitting diode (SLED). In certain embodiments, the light source 105 has a spectral profile centered in the short wave infrared spectrum, which ranges from wavelengths of about 900 nm to 1800 nm. In such embodiments the light source 105 may have a spectral profile centered at a wavelength of approximately 1300 nm. For example, the light source 105 may be a SLED centered at a wavelength of approximately 1310 nm and having a 56 nm full-width half-maximum.

The scan head 107 directs light from the light source 105 to the specimen 103 and receives light reflected from the specimen 103. For certain types of specimens, and depending upon the spectrum of the light source 105, the reflected light is reflected off the surface of the specimen 103. In certain embodiments, depending upon the specimen 103 being imaged, the scan head 107 may receive light reflected from multiple surfaces on or within the specimen 103. In such embodiments, as is discussed further below, the subsurface of the specimen 103 may be included as part of the produced image. In certain embodiments, the scan head 107 may include a micro electro-mechanical beam scanner. In such embodiments, the scan head 107 may be a dual-axis micro electro-mechanical scanning mirror with an integrated strain sensor for providing scan position feedback. Such a micro electro-mechanical scanning mirror may incorporated into a compact wand or scan arm (not shown), which facilitates placing the scan head adjacent tissues within the oral cavity, aural cavity, other body cavities, and within any other type of confined space to obtain images of a specimen.

The imaging system 101 also includes an interferometer 109 and a scanning spectrometer 111, both of which are optically coupled together and to the scan head 107. The interferometer 109 receives and collinearly couples light from the light source 105 and light reflected from the surface of the specimen. As those of skill in the art will recognized, the light from the light source 105 travels along the reference arm of the interferometer 109, and light reflected from the surface of the specimen, which is received through the scan head 107, travels along the sample arm of the interferometer 109. The interferometer 109 produces an interference output from the collinearly coupled light, and the interference output is directed to the scanning spectrometer 111. In certain embodiments, the interferometer 109 is a low coherence interferometer. In certain embodiments, the interferometer may be a Michelson interferometer, which may have a compact form and may be incorporated into a compact wand or scan arm.

The scanning spectrometer 111 uses a miniature Czerny-Turner monochromator setup to generate a scanned output from the interference output of the interferometer 109. The scanned output has a sub-spectrum of light which is narrower than, and within, the spectrum of light generated by the light source 105. The scanning spectrometer 1 1 1 creates a plurality of sub-spectrum from the interference output, with the sub-spectrum starting at a predetermined lower end and stopping at a predetermined upper end, all within the spectrum of light generated by the light source 105. The scanning spectrometer 111 sweeps through the plurality of sub-spectrum, such that each of the plurality of sub-spectrum, one sub-spectrum at a time, forms the scanned output of the scanning spectrometer 111. When one sweep ends, another begins, so that the scanning spectrometer 111 cyclically sweeps through the spectrum of light generated by the light source 105, one sub-spectrum at a time. As will be discussed further below, in certain embodiments the scanning spectrometer 111 may include a micro-mechanical device, so that the scanning spectrometer 111 may also be constructed in a compact manner.

The imaging system 101 also includes a detector 113 and a processor 115 operatively coupled together through an electrical connection. The detector 113 is optically coupled to the scanning spectrometer 111 so that the scanned output generated by the scanning spectrometer 111 is incident upon the detector 113. In response to the incident scanned output, the detector 113 generates a detector signal, and that detector signal is directed to the processor 115. In certain embodiments, the detector 113 may be constructed from a single point detector. Such an embodiment is enabled through the use of the scanning spectrometer 111, and the inclusion of a single point detector further enables the imaging system 101 to have a compact form. For embodiments in which short wave infrared is used, the single point detector may be an InGaAs point detector. Such point detectors, even though the sensing surface is formed from InGaAs, can be sufficiently inexpensive and compact so that the detector 117 may also be incorporated into a compact wand or scan arm. For example, InGaAs point detectors are available on the market which do not require any cooling, such that the point detector can be significantly more compact than a detector that requires cooling, as do most InGaAs array detectors.

In certain embodiments, the light source 105, the scan head 107, the interferometer 109, the scanning spectrometer 111, and the detector 113 form a data acquisition subsystem for the imaging system 101. Due to the compact nature of the components, such a data acquisition subsystem may be integrated into a compact wand or scan arm.

The processor 115 is programmed to process the detector signal to produce the image of the specimen. The manner in which the detector signal is processed are described in further detail below. The processor 115 is operatively coupled to the light source 105 and the scan head 105, and the processor 115 is also programed as a controller, such that the processor 115 controls operation of the light source 105 and the scan head 105. In certain embodiments, the processor 115 may be a field programmable gate array (FPGA). In still other embodiments, the processor 115 may be a system on a chip (SOC). In either of the aforementioned embodiment options, the processor 115 is able to perform all the necessary signal processing and still be economical and compact. In yet other embodiments, the processor 115 may be any other type of programmable device, not to be limited unless expressly stated in the claims.

It is anticipated that such an improved imaging system 101, due to the fact that it may be constructed in a compact design, may be used for a wide variety of applications, within a variety of medical-related disciplines, to image all types of specimen. The compact design of the imaging system 101 may prove to be particularly advantageous for enabling OCT imaging in personal care (i.e., non-clinical) and point-of-care (i.e., clinical) environments. The types of specimen that may be imaged include, without limitation, hard and soft tissue of the human body (e.g., oral tissue, skin tissue, etc.), tissue that is partially transmissive to light in spectral range produced by the light source 105, and just about any type of solid or semi-solid organic material. The imaging system 101 may also be used to image a specimen which is formed from inorganic material. Specific examples of applications of the imaging system 101 include in-vitro biological imaging, medical imaging such as ophthalmic imaging, dental imaging (intra-oral hard and soft tissue imaging), artificial skin, human skin, and animal skin imaging, and imaging for the detection of oral cancer and melanoma skin cancer. Such an improved imaging system 101 may also find additional applications in industrial fields of endeavor, such as testing of the thickness of layers, the size and distribution of pores, the integrity of fibers and the evaluation of materials such as plastic, rubber, and polymers, among many potential others. Moreover, it is anticipated that the improved imaging system 101 may be sufficiently compact and economical to be used at home. Such home use can display images to the user, or alternatively, the images may be uploaded to a cloud server for access and review by a medical professional.

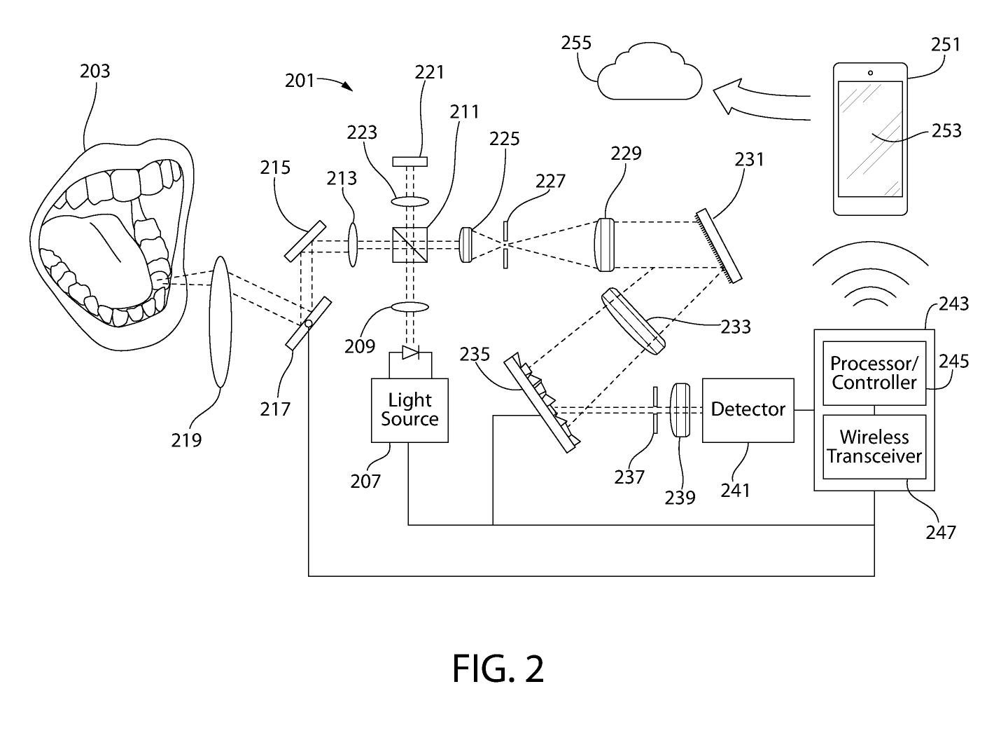

FIG. 2 illustrates an imaging system 201 positioned to image oral tissue, such as teeth, within an oral cavity 203. Soft oral tissue within the oral cavity 203 may also be imaged using the imaging system 201. A light source 207 generates light in a predetermined spectrum and directs the light through a focusing lens 209 to a beam splitter 211. In this embodiment, the light source 207 may be a broadband light source, such as a superluminescent light emitting diode (SLED). In certain embodiments, the light source 207 has a spectral profile centered in a short wave infrared spectrum. The light source 207 is operationally coupled to the data processing subsystem 243, which controls the emission of light from the light source 207.

The beam splitter 211 directs light from the light source 207 toward a focusing lens 213 and a folding mirror 215, which directs the light the beam scanner 217. The beam scanner 217 is controllable to direct light from the light source 207 through a focusing lens 219, so that light from the light source may be scanned along the surface of the specimen. In certain embodiments, the beam scanner 217 may be a dual-axis micro electro-mechanical scanning mirror with an integrated strain sensor for providing scan position feedback. The beam scanner 217 is operationally coupled to the data processing subsystem 243, which controls the beam scanner 217 to control the scan position of light from the light source 207 on the specimen.

Light from the light source 207 which is reflected by the specimen passes back through the focusing lens 219 and is incident on the beam scanner 217. The reflected light is directed by the beam scanner 217 to the folding mirror 215, through the focusing lens 213, and to the beam splitter 211. Light from the light source 207 is also directed by the beam splitter through the focusing lens 223 to the mirror 221, from which light is reflected back through focusing lens 223 to the beam splitter 211. At the beam splitter 211, light reflected from the mirror 221 is collinearly coupled with light reflected from the specimen to form the interference output, which is directed through the imaging lens 225, and the imaging lens 225 images the interference output onto the entrance slit 227 of the scanning spectrometer. In certain embodiments, the size of the entrance slit 227 may be chosen to balance wavelength resolution with signal-to-noise ratio (SNR) depending on the desired specifications of the imaging system 201.

In the scanning spectrometer, the interference output is collimated by a collimating lens 229 and directed to a reflective diffraction grating 231. In certain embodiments, the scanning spectrometer may also include a band pass filter to limit light incident on the diffraction grating 231 to the spectral range of the SLED. The diffraction grating 231 disperses the interference output horizontally into a plurality of sub-spectrum and directs the dispersed interference output through a focusing lens 233 and to a digital micro mirror 235. The amount of dispersion of the interference output is such that a lower end of the spectrum of the light source 207 is placed at one side of the digital micro mirror 235, while an upper end of the spectrum of the light source 207 is placed at the other side of the digital micro mirror 235. The diffraction grating 231 and the digital micro mirror 235 are optically arranged so that each sub-spectrum of the dispersed interference output is directed to one column of the reflective elements of the digital micro mirror 235. As is known in the art, the digital micro mirror 225 is a micro-mechanical device which includes an array of hundreds of thousands to millions of tiny micro-mirrors which can be independently rotated ±10-12°. For use in the scanning spectrometer, the micro-mirrors of the digital micro mirror 225 is controlled such that columns of the micro-mirrors are controlled as a unit, independently of the other columns of the micro-mirrors. The columns of micro-mirrors are independently controlled to direct light incident on the micro-mirrors of each respective column in a direction independent of each of the other columns. Thus, the digital micro mirror 235 is controllable to selectively direct a single sub-spectrum of the interference output to the output slit 237 of the scanning spectrometer. The digital micro mirror 235 is operationally coupled to the data processing subsystem 243, which controls the rotational positions of the micro-mirrors of the digital micro mirror 235. With the position of the micro-mirrors being controlled by the processing subsystem 243, the digital micro mirror 235 may generate the scanned output by directing the plurality of sub-spectrum, one sub-spectrum at a time, through the exit slit 237 and the focusing lens 239, and toward the detector 241.

The detector 241 detects the scanned output incident upon its face from the digital micro mirror 235 and generates a detector signal in response to the scanned output. That detector signal is passed to the data processing subsystem 243, which produces an image of the specimen from the detector signal. In certain embodiments, the detector 241 may be constructed from a single point detector. For embodiments in which short wave infrared is used, the single point detector may be an triGaAs point detector. The data processing subsystem 243 includes a processor 245 which may be programmed to process the detector signal to create an image of the specimen. The detector 241 may also include an analogue to digital converter to convert the analogue detector signal into a digital signal that may be analyzed by the processor 245. In certain embodiments, the analogue to digital converter may instead be included as part of the data processing subsystem 243.

The data processing subsystem 243 also includes a wireless transceiver 247 operationally coupled to the processor 245. The processor 245 may be programmed to transmit the image of the specimen using the wireless transceiver 247 to a remote device 251, which includes a display screen 253 for displaying the image. The wireless transceiver 247 may utilize any appropriate wireless protocol, such as WiFi or Bluetooth, with the wireless protocol not to be limited unless expressly stated in the claims. The remote device 251 may be any suitable type of programmable device, such as a desktop or laptop computer, smart phone, tablet, PDA, and the like. The remote device 251 is not to limit the claimed invention unless otherwise expressly stated in the claims. In certain embodiments, the processor 245 may communicate the digitized detector signal directly to the remote device 251. Although the imaging system 201 shows only a single remote device 251, in certain embodiments the processor 245 may communicate images and data to more than one remote device 251. In such embodiments, the processor 245 may communicate the image to one remote device, and the digitized detector signal directly to another remote device.

The remote device 251 may also communicate with a cloud server 255 using one or more public or private local area networks (LAN) and/or wide area networks (WAN). In certain embodiments, the remote device 251 may communicate one or more of the image or digitized detector signal data, along with any meta data associated with the image or digitized detector signal data, with the cloud server 255. In certain embodiments, the cloud server 255 may be used to store historical data associated with images of the specimen. In still other embodiments, the cloud server 255 may be used as a data aggregator, and the cloud server 255 may be used to perform additional data analysis, both on images and on digitized detector signal data.

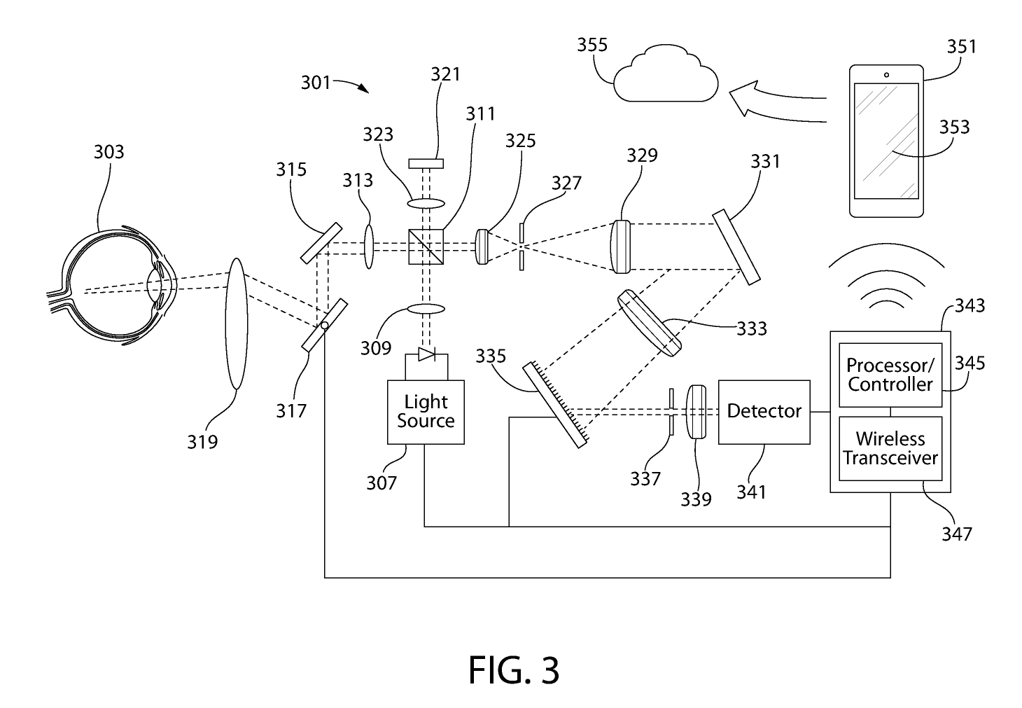

FIG. 3 illustrates an imaging system 301 positioned to image ocular tissue, such as the cornea and lens of an eye 303. A light source 307 generates light in a predetermined spectrum and directs the light through a focusing lens 309 to a beam splitter 311. In this embodiment, the light source 307 may be a broadband light source, such as a superluminescent light emitting diode (SLED). In certain embodiments, the light source 307 has a spectral profile centered in a short wave infrared spectrum. The light source 307 is operationally coupled to the data processing subsystem 343, which controls the emission of light from the light source 307.

The beam splitter 311 directs light from the light source 207 toward a focusing lens 313 and a folding mirror 315, which directs the light the beam scanner 317. The beam scanner 317 is controllable to direct light from the light source 307 through a focusing lens 319, so that light from the light source may be scanned along the surface of the specimen. In certain embodiments, the beam scanner 317 may be a dual-axis micro electro-mechanical scanning mirror with an integrated strain sensor for providing scan position feedback. The beam scanner 317 is operationally coupled to the data processing subsystem 343, which controls the beam scanner 317 to control the scan position of light from the light source 307 on the specimen.

Light from the light source 307 which is reflected by the specimen passes back through the focusing lens 319 and is incident on the beam scanner 317. The reflected light is directed by the beam scanner 317 to the folding mirror 315, through the focusing lens 313, and to the beam splitter 311. Light from the light source 307 is also directed by the beam splitter through the focusing lens 323 to the mirror 321, from which light is reflected back through focusing lens 323 to the beam splitter 311. At the beam splitter 311, light reflected from the mirror 321 is collinearly coupled with light reflected from the specimen to form the interference output, which is directed through the imaging lens 325, and the imaging lens 325 images the interference output onto the entrance slit 327 of the scanning spectrometer. In certain embodiments, the size of the entrance slit 327 may be chosen to balance wavelength resolution with signal-to-noise ratio (SNR) depending on the desired specifications of the imaging system 301.

In the scanning spectrometer, the interference output is collimated by a collimating lens 329 and directed to a collimating mirror 331. In certain embodiments, the scanning spectrometer may also include a band pass filter to limit light incident on the collimating mirror 331 to the spectral range of the SLED The collimating mirror 331 directs the interference output to a reflective diffraction grating 335. The diffraction grating 335 disperses the interference output horizontally into a plurality of sub-spectrum, and the diffraction grating 335 is pivotable to selectively direct a single sub-spectrum of the interference output to the output slit 337 of the scanning spectrometer.

The diffraction grating 335 is operationally coupled to the data processing subsystem 343, which controls the pivot position of the diffraction grating 335. By controlling the pivot position of the diffraction grating 335, pivoting of the diffraction grating 335 may generate the scanned output by directing the plurality of sub-spectrum, one sub-spectrum at a time, through the exit slit 337 and the focusing lens 339, and toward the detector 341.

The detector 341 detects the scanned output incident upon its face from the digital micro mirror 335 and generates a detector signal in response to the scanned output, and that detector signal is passed to the data processing subsystem 343. In certain embodiments, the detector 341 may be constructed from a single point detector. For embodiments in which short wave infrared is used, the single point detector may be an InGaAs point detector. The data processing subsystem 343 includes a processor 345 which may be programmed to process the detector signal to create an image of the specimen. The detector 341 may also include an analogue to digital converter to convert the analogue detector signal into a digital signal that may be analyzed by the processor 345. In certain embodiments, the analogue to digital converter may instead be included as part of the data processing subsystem 343.

The data processing subsystem 343 also includes a wireless transceiver 347 operationally coupled to the processor 345. The processor 345 may be programmed to transmit the image of the specimen using the wireless transceiver 347 to a remote device 351, which includes a display screen 353 for displaying the image. The wireless transceiver 347 may utilize any appropriate wireless protocol, such as WiFi or Bluetooth, not to be limited unless expressly stated in the claims. The remote device 351 may be any suitable type of programmable device, such as a desktop or laptop computer, smart phone, tablet, PDA, and the like. The remote device 351 is not to limit the claimed invention unless otherwise expressly stated in the claims. In certain embodiments, the processor 345 may communicate the digitized detector signal directly to the remote device 351. Although the imaging system 301 shows only a single remote device 351, in certain embodiments the processor 345 may communicate images and data to more than one remote device 351. In such embodiments, the processor 345 may communicate the image to one remote device, and the digitized detector signal directly to another remote device.

The remote device 351 may also communicate with a cloud server 355 using one or more public or private local area networks (LAN) and/or wide area networks (WAN). In certain embodiments, the remote device 351 may communicate one or more of the image or digitized detector signal data, along with any meta data associated with the image or digitized detector signal data, with the cloud server 355. In certain embodiments, the cloud server 355 may be used to store historical data associated with images of the specimen. In still other embodiments, the cloud server 355 may be used as a data aggregator, and the cloud server 355 may be used to perform additional data analysis, both on images and on digitized detector signal data.

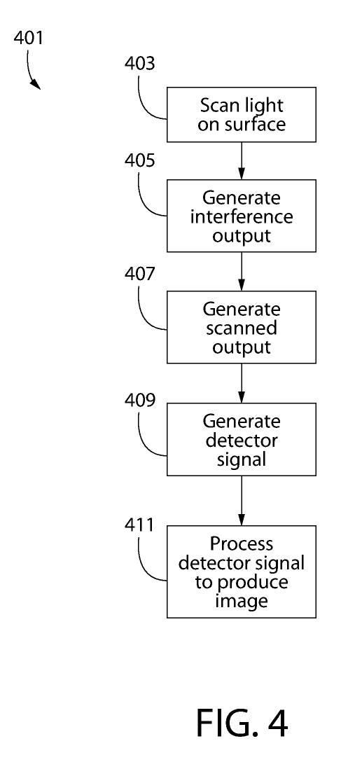

The process of creating an image of a specimen using OCT is shown in the flowchart 401 of FIG. 4. The programmable processors described above in connection with embodiments of the invention may be programmed to follow the process of the flowchart 401. In addition, the capabilities and parameters of the embodiments described above may be incorporated into the process of the flowchart 401. In certain embodiments, the processes shown and described herein may performed by a plurality of processors, with each processor being programmed to perform only a portion of the process, and with all the processors together being programmed to perform the entirety of the process.

The first step 403 of the process is to scan light from a light source on the surface of a specimen. Using light reflected from the specimen and light from the light source, the next step 405 is to generate an interference output using an interferometer. The interference output is directed into a scanning spectrometer, and in the next step 407 a scanned output is generated by the scanning spectrometer. In certain embodiments, the scanned output is generated using the any of the scanning spectrometer systems described herein. The process of creating the image of a specimen, however, is not to be so limited unless otherwise expressly stated in the claims. In the next step 409, the scanned output is used to generate a detector signal. In the last step 411, the detector signal is processed to produce an image of the specimen. The following details the process of producing the image from the detector signal.

As indicated above, the interference output is directed to the detector through the scanning spectrometer. The interference signal encodes depth resolved information from the specimen, and the interference signal as detected by the point detector can be expressed as:

I(k)=I_{S}(k)+I_{R}(k)+2\sqrt{I_{S}(k)I_{R}(k)}Σₙ αₙ cos(2kzₙ), (1)

where I_{R}(k) is the wavelength dependent intensity of light reflected from the reference arm of the interferometer, I_{S}(k) is the wavelength dependent intensity of reflected light reflected from the sample arm of the interferometer, k is the wave number, Zₙ is a depth within the specimen, and αₙ is the square root of the specimen reflectivity at the depth Zₙ. The third term on the right side of Eq. 1 represents the interference between the back reflected lights from the sample and reference arms of the interferometer.

In order to reconstruct the depth information, the spectrum represented by the interference signal I(k) is subjected to an inverse Fourier transform, which yields the following convolution:

|FT⁻¹[I(k)]|²=Γ²(z)⊗[I_{R}δ(z)+ΣₙI_{S,n}δ(z)+Σₙ αₙ²δ(z−zₙ)+Σₙ αₙ²δ(z+zₙ)], (2)

where Γ(z) represents the envelope of the coherence function. The first and second terms on the right hand side of Eq. 2 describe the autocorrelation (or self-interference) of light from the reference arm of the interferometer and light from the sample arm of the interferometer, respectively. The third and fourth terms on the right hand side of Eq. 2 are due to the interference of back reflected light from reference and sample arms of the interferometer and the complex conjugates thereof.

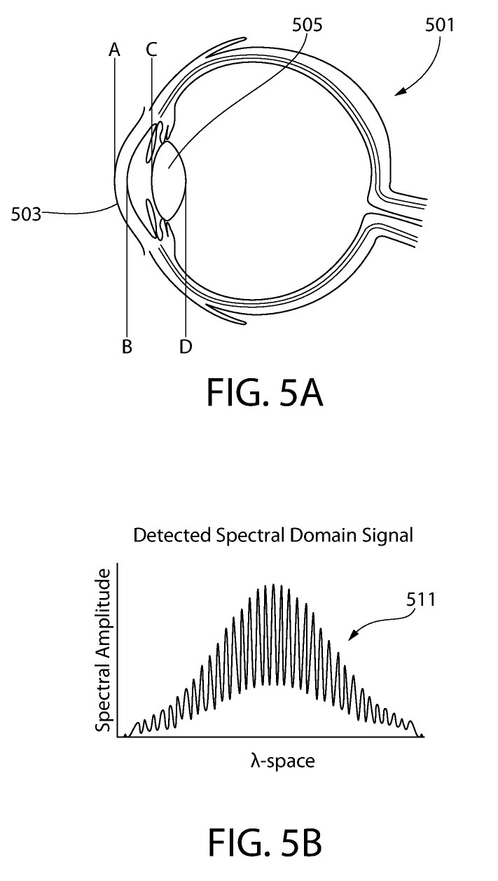

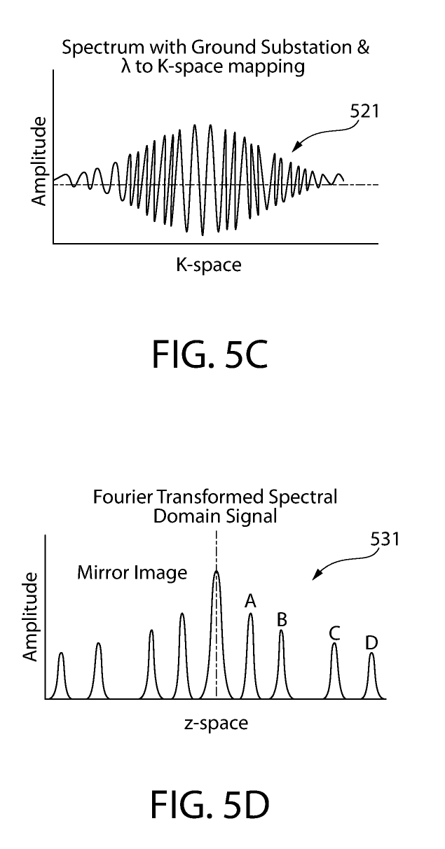

FIG. 5A shows an image of a human eye 501, which may be scanned by the scanning system described herein. The eye 501 includes a cornea 503 and a lens 505. The cornea 503 has an anterior surface A and a posterior surface B, while the lens 505 has an anterior surface C and a posterior surface D. Both the cornea 503 and the lens 505 are transmissive to light in the SWIR spectrum. During the scan, data is collected by scanning in two axial directions, along an x-axis and a y-axis, where these two axes are orthogonal to a z-axis, which extends into the specimen. FIG. 5B shows an example of the intensity spectrum 511 detected by the detector during a scan of the eye 501, and is representative of a detector signal for both axial scans. The intensity spectrum of FIG. 5B is shown in wavelength space (λ-space). The detected intensity spectrum is converted to digital data by an analogue to digital converter for further processing.

In order to suppress autocorrelation, self-cross correlation, and camera noise artifacts, first, all the data from spectrally resolved interference signals for scans along one of the scan axes are ensemble-averaged for each sub-spectrum to obtain a reference spectrum. The reference spectrum is then subtracted from the spectrally resolved interference signals for scans along the other scan axis. In performing the Fourier transform on the λ-space spectrally resolved interference signals, the physical distance along the z-axis (which is in coordinate space, also called z-space) is related to the wave number indirectly, as k=2π/λ, which tends to result in an inaccurate depth profile along the z-axis. In order to obtain a proper depth profile along the z-axis, the subtracted spectrally resolved interference signals are first remapped from λ-space to wavenumber space (k-space) by use of the spline interpolation method, FIG. 5C shows an example of a spectrally resolved interference signal remapped into k-space 521. Now, by performing a Fourier transform on the spectrally resolved interference signals remapped into k-space, the physical distance along the z-axis in z-space is now directly related to k-space. The results of this Fourier transform therefore yields a proper depth profile along the z-axis. FIG. 5D shows an example of the results 531 of a Fourier transform, in z-space, performed on the spectrally resolved interference signals remapped into k-space. As can be seen, the data shows peaks along the z-axis for the anterior surface A and the posterior surface B of the cornea 503, along with the anterior surface C and the posterior surface D of the lens 505.

During actual use of an imaging system, each sweep of the interference output can result in up 512, 1024, or more spectral sampling points captured at the detector. In certain embodiments, the output of the detector may be subjected to a band pass filter prior to being converted to digital data. Each line of digital data, resulting from digitization of detector data from one sub-spectrum, is independently acquired and processed by the data processing sub-system. The image may be constructed by collating a plurality of the lines of digital data together to form a single image frame using consumer-level frame grabber software, which is also capable of processing the lines of digital data in batches. As should be appreciated, the components for processing the scan data obtained from the image acquisition subsystem can be quite economical in terms of cost and power consumption. Therefore, the overall system is appropriate for both prosumer and consumer uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

1. An imaging system comprising:
a scan head optically coupled to a light source and configured to scan light from the light source onto a portion of a specimen and to receive light reflected from the portion of the specimen;
an interferometer optically coupled to the light source and to the scan head and configured to generate an interference output using light from the light source and light reflected from the portion of the specimen;
a scanning spectrometer optically coupled to the interferometer to receive the interference output and configured to generate a scanned output, the scanned output having a sub-spectrum of light which is narrower than a spectrum of light generated by the light source; and
a detector optically coupled to the scanning spectrometer to detect the scanned output and configured to generate a detector signal from the detected scanned output.

2. (canceled)

3. The imaging system of claim 1, wherein the light source has a spectral profile centered in a short wave infrared spectrum.

4. The imaging system of claim 1, wherein the light source comprises a broadband superluminescent light emitting diode.

5. The imaging system of claim 1, wherein the scan head comprises a micro electro-mechanical beam scanner directing light from the light source onto the portion of the specimen and receiving light reflected from the portion of the specimen.

6. The imaging system of claim 1, wherein the interferometer comprises a low coherence interferometer.

7. The imaging system of claim 1, wherein the scanning spectrometer comprises:
a diffraction grating on which the interference output is incident; and
a digital micro mirror optically coupled to the diffraction grating;
wherein the diffraction grating disperses the interference output into a plurality of sub-spectrum and directs the dispersed interference output to the digital micro mirror; and
wherein the digital micro mirror is controlled by a programmable processor to generate the scanned output by directing the plurality of sub-spectrum, one sub-spectrum at a time, toward the detector.

8. The imaging system of claim 1, wherein the scanning spectrometer comprises a micro-machined diffraction grating on which the interference output is incident, wherein the diffraction grating disperses the interference output and is pivotable, and wherein the pivoting of the diffraction grating is controlled by a programmable processor to generate the scanned output by directing a plurality of sub-spectrum, one sub-spectrum at a time, toward the detector.

9. The imaging system of claim 1, wherein the detector comprises a point detector.

10. The imaging system of claim 1, further comprising a processor operatively coupled to the detector to receive the detector signal, the processor programmed to generate an image of the portion of the specimen from the detector signal.

11. (canceled)

12. The imaging system of claim 10, further comprising a display screen operatively coupled to the processor to receive and display the image.

13.-18. (canceled)

19. An imaging method comprising:
scanning light from a light source onto a portion of a specimen;
generating an interference output by collinearly coupling light from the light source and reflected light from the portion of the specimen;
generating a scanned output using a scanning spectrometer by dispersing the interference output into a plurality of sub-spectrum within the predetermined spectrum, and by cycling through each of the sub-spectrum to form the scanned output;
generating a detector signal from the scanned output; and
processing the detector signal to generate an image of the portion of the specimen.

20.-34. (canceled)

35. An imaging system comprising:
a data acquisition subsystem comprising:
a light source producing light in a predetermined spectrum;
a scan head optically coupled to the light source and configured to scan light from the light source onto a portion of a specimen and to receive light reflected from the portion of the specimen;
an interferometer optically coupled to the light source and to the scan head to receive, respectively, light from the light source and light reflected from the portion of the specimen, the interferometer configured to generate an interference output by collinearly coupling light from the light source and light reflected from the portion of the specimen;
a scanning spectrometer optically coupled to the interferometer to receive the interference output and configured to disperse the interference output into a plurality of sub-spectrum within the predetermined spectrum, wherein the scanning spectrometer generates a scanned output by cycling through each of the sub-spectrum; and
a detector optically coupled to the scanning spectrometer to detect the scanned output, the detector configured to generate a detector signal from the detected scanned output; and
a data processing subsystem comprising:
a processor operatively coupled to the detector to receive the detector signal, the processor programmed to generate an image of the portion of the specimen from the detector signal; and
a wireless transceiver operatively coupled to the processor, wherein the processor is programmed to transmit the image using the wireless transceiver.

36. The system of claim 35, further comprising a remote device wirelessly coupled to the data processing subsystem to receive the image and comprising a display screen, wherein the remote device is programmed to display the image on the display screen.

37. The system of claim 36, wherein the remote device is communicably coupled with a server over a network and is programmed to transmit the image to the server.

38. (canceled)

39. The imaging system of claim 35, wherein the light source has a spectral profile centered in a short wave infrared spectrum.

40. (canceled)

41. The imaging system of claim 35, wherein the scan head comprises a micro electro-mechanical beam scanner directing light from the light source onto the portion of the specimen and receiving light reflected from the portion of the specimen.

42. The imaging system of claim 35, wherein the interferometer comprises a low coherence interferometer.

43. The imaging system of claim 35, wherein the scanning spectrometer comprises:
a diffraction grating on which the interference output is incident; and
a digital micro mirror optically coupled to the diffraction grating;
wherein the diffraction grating disperses the interference output into the plurality of sub-spectrum and directs the dispersed interference output to the digital micro mirror; and
wherein the digital micro mirror is controlled by a programmable processor to generate the scanned output by directing the plurality of sub-spectrum, one sub-spectrum at a time, toward the detector.

44. The imaging system of claim 35, wherein the scanning spectrometer comprises a micro-machined diffraction grating on which the interference output is incident, wherein the diffraction grating disperses the interference output and is pivotable, and wherein the pivoting of the diffraction grating is controlled by a programmable processor to generate the scanned output by directing the plurality of sub-spectrum, one sub-spectrum at a time, toward the detector.

45. The imaging system of claim 35, wherein the detector comprises a point detector.

46.-51. (canceled)
